(19) United States
(12) Patent Application Publication
(10) Pub. No.: US 2010/0065048 A1
(43) Pub. Date: Mar. 18, 2010

(54) DRY POWDER INHALER DEVICES

(75) Inventors: Rudi Mueller-Walz, Schopfheim (DE); Thomas Eggimann, Bubendorf (CH)

Correspondence Address: MINTZ, LEVIN, COHN, FERRIS, GLOVSKY AND POPEO, P.C, ONE FINANCIAL CENTER, BOSTON, MA, 02111, US

(73) Assignee: JAGOTEC AG, Muttenz (CH)

(21) Appl. No.: 12/223,957

(22) PCT Filed: Feb. 7, 2007
(86) PCT No.: PCT/EP2007/001031

§ 371 Date: Nov. 30, 2009

(30) Foreign Application Priority Data
Feb. 13, 2006 (GB) 0602897.1

(51) Int. Cl. A61M 15/00

(52) U.S. Cl. 128/203.15

(57) ABSTRACT

A DPI device comprising a dispensing chamber for receiving a discrete dose of medicament-containing powder and means for delivering said dose from the dispensing chamber to a patient in an air-flow that passes from the chamber to the patient via a mouth-piece along a first air passage that comprises de-agglomerating means, the device additionally comprises a second air passage in fluid communication with the dispensing chamber and the mouth-piece and which by-passes the de-agglomerating means and which is located such that it receives a portion of said air-flow that is free, or substantially free, of powder.

### RELATED APPLICATIONS

This application is a national stage application, filed under 35 U.S.C. §371, of International Application No. PCT/EP2007/001031, filed on Feb. 7, 2007, which claims the benefit of GB 0602897.1, filed Feb. 13, 2006.

The present invention is concerned with improvements in dry powder inhaler devices, and in particular improvements in breath-actuated dry powder inhaler devices, for delivering pharmaceutical substances to the respiratory tracts of patients.

Asthma and other respiratory diseases have long been treated by inhalation of a medicament. Medicaments may be presented in the form of a solution or suspension in an aerosol propellant and delivered from a pressurized metered dose inhaler. Alternatively, they may be presented in the form of dry powders alone or mixed with suitable carrier materials, such as lactose. The present disclosure is concerned with devices for delivering dry powders, so-called Dry Powder Inhalers or DPIs.

DPIs may be used to dispense either individual pre-measured doses of medicament-containing powder that are inserted into a dispensing chamber before use or successive metered doses of powder contained in a bulk reservoir which can be transferred to a dispensing chamber. DPIs contain an air passage that passes over or through a dispensing chamber that is in fluid communication with an exit port adapted to be inserted into the mouth and or nasal passage of a patient. Upon actuation of a DPI device, an air-flow is generated that passes through the air passage and over the dispensing chamber thereby to entrain the powder dose and carry it on a stream of air through the exit port and into the respiratory tract of a patient.

It is very often the case that the powders contained in DPI devices contain particles in agglomerated states, that is, powders are comprised of agglomerated particles of medicament or agglomerated particles of medicament and carrier. In order that the medicament particles can pass deep into the respiratory tract of a patient, it is deemed necessary to separate these agglomerates as far as possible before the powder is inhaled by a patient.

To assist in the process of de-agglomeration, DPIs typically contain mechanical means that disrupt the air flow as it passes through the device. These mechanical means are provided in order to generate turbulent air flow that exerts shear forces on the agglomerated powder particles by promoting collisions between particles and between particles and walls of the air passage. Suitable means may comprise a series of bends or constrictions provided in an air passage used to direct the air-flow. Additionally, or alternatively the air passage may contain a series of inter-digitated baffles, or any combination of these features that have the effect of rendering the air path more turbulent.

It is desirable that the process of de-agglomeration occurs at a site proximate to the exit port of the mouth-piece, and indeed in many DPI devices the mechanical means are usually provided in a mouth-piece attached to, or adapted to be attached to, a DPI device.

In some DPI devices an air-flow is generated from a source of pressurized gas. However, this approach requires synchronization of a patient's inhalation effort with the hand actuation of the pressurized gas source. Unfortunately, many patients experience hand-mouth co-ordination problems that can result in their receiving inaccurate doses of medicament. Accordingly, DPI devices have been developed that utilize the action of a patient's inhalation to both entrain and de-agglomerate powders to overcome this co-ordination problem. These devices are often referred to simply as breath-actuated devices.

Breath-actuated devices rely on the inspiratory effort of a patient creating an under-pressure in the device, which draws air, external of the device, into an air passage internal of the device and over a powder dose held in a dispensing chamber. The air-flow entrains the powder and carries it through the exit port to be inhaled by the patient in the manner already described above.

Whereas breath-actuated devices are undoubtedly advantageous in that they avoid the synchronisation problems and promote more reliable dosing, the efficiency of powder entrainment and de-agglomeration in these devices depends solely upon the inspiratory effort of a patient. This effort may be very difficult to achieve in the case of severely compromised patients. Furthermore, de-agglomerating means, such as those described above, generate a certain resistance to air-flow, which requires of the patient an even greater inspiratory effort in order to inhale all of a metered dose. As such, there is a risk that highly compromised patients such as those experiencing an asthma attack or those suffering from COPD or CF would be unable to inhale all, or substantially all, of a dose metered by the device.

There remains a need to provide a DPI device, in particular a breath-actuated DPI device that is able not only to efficiently de-agglomerate a powder, but also to reduce the inspiratory effort required to inhale all or substantially all of a dose that is metered by the device.

Prior art devices have been developed that are provided with means for reducing resistance associated with de-agglomeration means. One example is a device marketed by GSK under the name TURBUHALER. Another device is marketed by Astra Zeneca under the name DISKUS. Whereas these devices look very different, they share common features: Both contain an air passage through which air is drawn into the device to create a primary air-flow that flows over a powder sample thereby to entrain the powder and carry it into the patient's respiratory tract via a mouth-piece. The generation of this primary air-flow is driven by the inspiratory effort of the patient in each case. Both devices also contain additional air-vents through which air, external of the device, is drawn into and through the mouth-piece. These additional air-vents create secondary air-flows are directed into the mouth-piece and are thought to interact with the primary air-flow to create turbulent air-flow that assists in the de-agglomeration process. It is also thought that these additional air-streams may reduce air-resistance at the mouth-piece making inhalation easier for the patient.

Whereas these prior art means may reduce the inspiratory effort required of a patient, a part of the patient's inspiratory effort is nevertheless diverted away from generating the primary air-flow and into creating the secondary air-flows. In these circumstances, particularly in the case of severely compromised patients, the primary air-flow may not be sufficient to efficiently entrain all of a metered dose and deliver it to the lungs of a patient.

The applicant has now developed means for reducing air resistance created by de-agglomeration means but which does not divert a patient's inspiratory effort away from the generation of a primary air-flow and as such does not reduce the efficiency of entrainment and de-agglomeration of powder.

Accordingly, the invention provides a DPI device comprising a dispensing chamber for receiving a discrete dose of medicament-containing powder and means for delivering said dose from the dispensing chamber to a patient in an air-flow that passes from the chamber to the patient via a mouth-piece along a first air passage that comprises de-agglomerating means, the device additionally comprises a second air passage in fluid communication with the dispensing chamber and the mouth-piece, by-passing the de-agglomerating means and being located such that it receives a portion of said air-flow that is free, or substantially free, of powder.

In a preferred embodiment, the invention provides in one of its aspects a DPI device comprising:

a dispensing chamber for receiving a discrete dose of medicament-containing powder;

means for delivering said dose on an air-flow from the dispensing chamber to a patient via a mouth-piece; said means for delivering a dose comprising an air passage in fluid communication between dispensing chamber and exit port, which is adapted to channel said air-flow through the device;

means for de-agglomerating a powder disposed along the air passage; and

means for bifurcating the air-flow into first and second portions, the first portion containing entrained powder that passes through the de-agglomerating means before being dispensed from an exit port in the mouth-piece, the second portion containing no entrained powder and by-passing the de-agglomerating means before being dispensed from the mouth-piece.

The invention provides in another aspect a method of de-agglomerating dry-powder from a DPI device prior to its inhalation by a patient, the method comprising:

generating an air-flow for entraining a powder;

bifurcating said air-flow into a first portion containing entrained powder, and a second portion not containing dry powder;

channeling said first portion through a mouth-piece and into the respiratory tract of a patient via means for de-agglomerating dry powder; and

channeling said second portion through the mouth-piece and into the respiratory tract of a patient by-passing said de-agglomerating means.

The present invention, in contra-distinction to the prior art referred to above, works on the principle of a patient's inspiratory effort creating only a single air-flow passing through the device. It is the same air-flow that both entrains the powder and passes out of the exit port of a mouth-piece and into the respiratory tract of a patient. The single air-flow is bifurcated by mechanical means. Because none of the patient's inspiratory effort is diverted into creating secondary air-flows drawn externally of the device, powder can be efficiently entrained even at relatively low inspiratory effort levels.

The means for bifurcating the air-flow may be provided in a mouth-piece attached to, or adapted to be attached to, a DPI device. The mouth-piece can be provided with two channels both of which are disposed in the path of the primary air-flow generated in the device. In this manner, the air-flow will be split into first and second portions passing respectively through first and second channels. Said first channel can contain means for de-agglomerating powder. Such means may simply consist of a series of bends in the channel. Additionally or alternatively the channel may contain inter-digitated baffles, or it may narrow and widen variably to constrict air-flow. By any of these means or a combination of them, entrained powder will experience turbulent air that will have the desired de-agglomerating effect. A second channel, not containing de-agglomerating means will receive a second portion of the air-flow which can flow through this chamber in an essentially linear manner and with lower air-resistance.

Each portion of the air-flow may pass through separate exit ports provided in a mouth-piece to be inhaled by a patient. Alternatively, the two channels may be in fluid communication such that each portion of the air-flow is re-combined down-stream of the de-agglomerator just before exiting a single exit port provided in the mouth-piece. The latter construction is a preferred embodiment of the invention in that the act of re-combining each portion of the air-flow may create additional turbulence and have the effect of further de-agglomerating the powder before it exits the mouth-piece. The two portions of air-flow can be re-combined simply by providing a small orifice in the wall separating the two channels down-stream of the de-agglomerating means and which brings both channels into fluid communication.

The size of the orifice may be adjusted in order to obtain a desired air-resistance through the mouth-piece. Typically, having regard for the size of DPI devices and in particular the size of a mouth-piece that can comfortably be accommodated in the mouth of a patient, the orifice may have a diameter of 0.05 to 6 mm, more preferably 2 to 2.5 mm. The skilled person will also appreciate that more than one orifice can be employed to the same effect.

The skilled person will also appreciate that whereas the invention has been described in terms of splitting the primary air-flow into two streams, it is possible to have more than two channels provided that one of them at least contains de-agglomerating means.

It is desirable that all, or substantially all, of the metered dose of powder passes through the channel containing de-agglomerating means. In order to ensure that this is the case, the DPI device is provided with means to position a metered dose in a position proximate to the entrance to the first channel containing de-agglomerating means. Upon actuation of the device, the generated air-flow will pass through the device in the direction of the mouth-piece and as it passes over the metered dose it will entrain the powder and carry it into the channel containing de-agglomeration means, and not the second channel.

By means of the present invention, the portion of the air-flow passing through said second channel will do so with essentially linear flow. In this way, air resistance is reduced and the patient will find it easier to inhale. Furthermore, because all of the entrained powder passes through de-agglomeration means, the powder dose emitted from the device will be highly de-agglomerated which should result in the emitted dose having a higher Fine Particle Fraction (FPF).

The fraction of the powder contained in the emitted dose that is of small enough aerodynamic diameter to reach the deep lung upon inhalation is often referred to as the fine particle fraction (or FPF) of the emitted dose. The absolute amount of fine particles emitted is often referred to as the Fine Particle Dose (or FPD).

A device of the present invention is capable of delivering a medicament to a patient with an emitted dose containing a high fine particle fraction based on the emitted dose, for example in excess of 15%, more particularly 25 to 75%. In particular, a device of the present invention is capable of emitting a dose of medicament to the respiratory tract of a patient wherein the fraction of particles having mean aerodynamic diameter of 4.4 microns or less as measured using an Anderson Cascade Impactor is in excess of 15% of the emitted dose, more particularly 25 to 75% of the emitted dose.

The emitted dose and its variance can be measured using a Dosage Unit Sampling Apparatus (DUSA). The FPF can be measured using an Andersen Cascade Impactor (ACI). The measurement methodology and the apparatus therefor are well known in the art, and are described in the United States Pharmacopoeia Chapter <601>, or in the inhalants monograph of the European Pharmacopoeia, both of which documents are hereby incorporated by reference. The USP states that the Apparatus 1 should be used for the measurement of FPF. The USP also states that Delivered dose Uniformity should be measured with DUSA or its equivalent. However, the Delivered dose and Delivered dose uniformity are preferably measured using the so-called Funnel Method. The Funnel Method is described in Drug Delivery to the Lungs, VIII p 116 to 119, which is hereby incorporated by reference. In summary, the Funnel Method consists of discharging a formulation from a DPI into a Funnel Apparatus, which basically consists of a standard Buchner Funnel. The discharged dose is captured on the glass sinter of the Funnel, and can be washed off, and the dose determined using HPLC analysis. The Funnel Method gives comparable results to the standard USP apparatus, and is generally considered to be an equivalent of the DUSA apparatus.

The present invention preferably relates to devices that are breath-actuated.

A problem with breath-actuated devices is that even at very low inspiration air-flow rates the process of entrainment will occur immediately when air begins to enter into the device and passes across a metered dose. Often, if a patient is naïve, or if the patient is severely compromised, a metered dose of powder can become entrained even though the air flow rate through the device is insufficient for the powder to be carried out of the device and into the respiratory tract of a patient. A solution to this problem resides in the use of valve means that will restrict air flow through the device until such time as sufficient inspiratory effort is achieved to entrain a metered dose and drive it through the device and into the respiratory tract of a patient.

As stated above, valve means work by only permitting sufficient air to be drawn into a device to both entrain a powder dose and drive it into the respiratory tract of a patient once a patient's inspiratory effort reaches a pre-determined minimum exertion level. The minimum inspiratory effort is set having regard to such factors as the size of a metered dose, and the nature and severity of a patients disease, such that upon actuation of the valve means the air drawn into the device is of sufficient flow rate that it can efficiently entrain a metered dose and deliver it to a patient's lungs. Essentially, the valve means ensures that all, or substantially all, of the metered dose is emitted from the device to be inhaled by a patient. It is highly undesirable that a patient inhales only to leave medicament in the device or in his or her oral cavity, and not delivered to the lung. Valve-containing breath-actuated devices guard against this outcome and as such represent a preferred embodiment of the present invention.

The invention described herein is highly advantageous when used in conjunction with a device that operates by drawing an air-flow through the device via valve means. This is because all of a patient's inspiratory effort can be directed at achieving the minimum actuation flow rate needed to open the valve. Should the prior art methods described above be employed in valve-actuated devices, it is unlikely that moderate to severely compromised patients would be able to inspire with sufficient force to actuate the valve given that a certain amount of their inspiratory effort is diverted to create the secondary air flows.

The minimum inspiratory flow rate needed to trigger the valve means (the “minimum actuation flow rate”) can be selected having regard to the nature of a patient and the type and severity of condition to be treated. Importantly, the actuation flow rate should not be so low that the valve can be actuated without sufficient flow rate to entrain and deliver to the respiratory tract of a patient all, or substantially all, of a dose metered into the dispensing chamber. Preferably, the minimum actuation flow rate should be about 30 or greater litres/minute, more particularly 30 to 60 litres/minute.

Accordingly, in a preferred embodiment of the present invention there is provided a breath-actuated inhaler device for dispensing a dose of powder to the respiratory tract of a patient, which device comprises:

a hollow housing containing an air inlet, and valve means in releasable sealing arrangement with said air inlet, the valve means being actuated to allow an air-flow to pass through an air passage in the housing in response to a pre-determined minimum inspiratory effort of a patient;

a dispensing chamber internal of the housing for receiving a discrete dose of medicament-containing powder; and

a mouth-piece attached, or adapted to be attached, to said housing, said mouth-piece comprising:

a hollow body having at a first end an exit port through which a powder can pass into the respiratory tract of a patient, and at a second end an opening in communication with the air-passage of the device when the mouth-piece is connected to the housing; and first and second channels bringing said exit port and opening into fluid communication, wherein said first channel describes a tortuous air passage for de-agglomerating a powder,

and wherein upon actuation said air-flow is bifurcated such that a first portion of the air-flow containing the entrained powder passes through the first channel and through an exit port into the respiratory tract of the patient, and a second portion of said air-flow not containing entrained dry powder passes through the second channel of the mouth-piece and through an exit port in the mouth-piece and into the respiratory tract of the patient.

In a more particular embodiment of the present invention there is provided a breath-actuated inhaler device for dispensing a powder to the respiratory tract of a patient comprising:

a hollow housing having openings at opposed ends, having a mouth-piece attached to the housing at a first end and valve means positioned inside the housing and covering a second end, the mouth-piece having first and second channels extending therethrough, said first channel describing a tortuous path for de-agglomerating powder entrained on an air-flow; a protective cap covering said mouth-piece, said cap being moveable between an closed position covering the mouth-piece and an open position exposing the mouth-piece for use by a patient;

a powder reservoir located inside the housing and including a funnel outlet;

a moveable dosing mechanism located inside the housing containing a dispensing chamber, said dispensing chamber being moveable between a receiving position directly under said funnel outlet, and a dispensing position proximate to the first channel of the mouth-piece when the cap is moved from closed to open position;

a slidable cover covering said dispensing chamber when in its dispensing position;

said valve means being moveable between a rest position sealing one opposed end opening, and a forward position in response to a defined minimum inspiratory effort during inhalation by a patient, said valve shield moving said slidable cover from over the dispensing chamber when moving into its forward position to permit an air-flow external of the device to enter the housing thereby to entrain the powder such that a first portion of said air-flow containing entrained powder passes through the first channel of the mouth-piece before passing through an exit port in the mouth-piece and into the respiratory tract of a patient, and a second portion of the air-flow not containing entrained dry-powder passes through the second channel and through an exit port in the mouth-piece to be inhaled by a patient;

returning means for returning said dispensing chamber to the receiving position after inhalation by a patient; and

recording unit positioned inside the housing, said recording unit recording the number of inhalation operations performed.

A valve-actuated device is described in U.S. Pat. No. 6,182,655. A particularly preferred embodiment of the present invention consists of the device disclosed in that patent duly modified such that the mouth-piece contains at least two air channels, wherein one contains a de-agglomerator, substantially as described herein. The U.S. Pat. No. 6,182,655 is incorporated herein in its entirety by reference.

U.S. Pat. No. 6,182,655 describes a dry powder inhaler device for the administration of a pharmacological powder. The device consists of a housing, which contains a reservoir holding a powder formulation and having an funnel outlet through which the formulation can be released, and a dosing mechanism comprising a dosing chamber adapted to receive a unit dose of the powder from the reservoir via the funnel outlet, which is moveable within the housing to transport a unit dose of powder formulation from a position directly under the reservoir funnel outlet to a position proximate to a mouth-piece where the powder can be entrained upon an air-flow upon actuation of the device by the inspiratory effort of a patient. In order to retain the powder in the dispensing chamber in its position proximate to the mouth-piece, a shutter is slidably fitted over the dispensing chamber.

Fitted over the mouth-piece is a cap that is connected to the housing. The cap covers the mouth-piece when the device is not in use. Connecting means between cap and dosing mechanism translates movement of the cap to the dosing mechanism, such that when the cap is opened to expose the mouth-piece, the dosing mechanism moves a unit dose in the dosing chamber in the manner described above. In this way, removal of the cap effectively loads the device in readiness for actuation by a user.

The device relies on gravity to feed the powder from the reservoir into the dosing chamber. To ensure that a unit dose is correctly and completely delivered to the dosing chamber, the device should be held in an orientation such that the reservoir funnel outlet sits vertically, or substantially vertically, above the dosing chamber. In order to prevent a user readying the device for actuation with no dose or an incomplete dose in the dosing chamber, the device is equipped with a gravity-actuated locking mechanism for the cap. Essentially this locking mechanism only permits removal of the cap when the device is held by a user in an orientation such that the reservoir funnel outlet is positioned vertically, or substantially vertically, above the dosing chamber. In the preferred embodiment described in the aforementioned patent, the reservoir exit port is held in the correct orientation with respect to the dosing chamber when the device is held such that both the housing and cap are held in the same, or substantially the same, horizontal plane. In such an orientation the cap can be removed and the device can be actuated to deliver correctly a unit dose. However, if the device is tilted or rotated out of this plane, then the gravity-actuated locking mechanism will prevent the cap being removed.

The device contains a moveable valve shield internally of the housing that sits over air-inlet formed in the housing and covers said inlet when the device is not in use. The valve shield is moveable from this rest position very slightly when the cap is removed. The valve shield is further moveable to a forward position in response to a defined inspiratory effort during inhalation by a patient. The valve shield is connected to the shutter over the dispensing chamber, and the movement of the valve shield into its forward position is communicated to the shutter such that the shutter is opened when the valve shield moves into its forward position. At the same time, the air-flow passing through the device entrains the powder and ejects it through the mouth-piece into the respiratory tract of a patient via de-agglomerating means designed to break up agglomerated powder particles.

Once particles of a dry powder have passed through de-agglomerating means it is desirable that they do not re-agglomerate before being inhaled by a patient. Accordingly, it is preferred if the de-agglomerating means are provided as close to the exit of the mouth-piece as possible. Indeed, it is preferred if de-agglomerating means are provided in the mouth-piece. The mouth-piece may be integral with the device or it may be separately formed and adapted to fit onto the device. Such a mouth-piece forms another aspect of the present invention.

Accordingly, in another aspect of the present invention there is provided a mouth-piece adapted to releasably attach to an inhaler device comprising:

a hollow body comprising:

at one end an exit port through which a dry powder can pass into the mouth of a patient, and at the other end an opening through which an air-flow emitted from the inhaler device may pass; and

means for releasably attaching the mouth-piece to an inhaler device;

the hollow body having running therethrough a first channel describing a tortuous air passage for de-agglomerating a powder passing through the channel entrained on a first portion of said air-flow; and

a second channel running therethrough, through which a second portion of said air-flow passes not containing entrained powder.

The inhaler device of the present invention can be used to store and administer all manner of pharmaceutically active agents. A non-limiting list of agents is provided below:

Any active substance useful in treating conditions of the lung, such as asthma or chronic obstructive pulmonary disease (COPD), or useful being administered through the lung to treat systemic disease states may be employed in containers of the present invention. Suitable active agents include: beta.2-adrenoreceptor agonists such, for example, as salbutamol, terbutaline, rimiterol, fenoterol, reproterol, adrenaline, pirbuterol, isoprenaline, orciprenaline, bitolterol, salmeterol, formoterol, clenbuterol, procaterol, broxaterol, picumeterol, TA-2005, mabuterol and the like and their pharmacologically acceptable esters and salts; steroids, including any of the materials selected from the group consisting of budesonide, ciclesonide, mometasone, fluticasone, beclomethasone, flunisolide, loteprednol, triamcinolone, amiloride and rofleponide or a pharmaceutically acceptable salt or derivative of these active compounds, such, for example, as mometasone furoate, fluticasone dipropionate, beclomethasone dipropionate, triamcinolone acetonide or flunisolide acetate (where optically active, these materials can be used in the form of their active isomer or as an isomer mixture); anticholinergic bronchodilators such, for example, as ipratropium bromide and the like; anti-allergic medicaments such, for example, as sodium cromoglycate and nedocromil sodium; expectorants; mucolytics; antihistamines; cyclooxygenase inhibitors; leukotriene synthesis inhibitors; leukotriene antagonists, phospholipase-A2 (PLA2) inhibitors, platelet aggregating factor (PAF) antagonists and prophylactics of asthma; antiarrhythmic medicaments, tranquilisers, cardiac glycosides, hormones, antihypertensive medicaments, antidiabetic-, such for example as insulin, antiparasitic- and anticancer-medicaments, sedatives and analgesic medicaments, antibiotics, antirheumatic medicaments, immunotherapies, antifungal and antihypotension medicaments, vaccines, antiviral medicaments, vitamins, anti-oxidants, free-radical scavengers; COX II inhibitors such as celecoxib; NSAIDS; PDE4 inhibitors and PDE5 inhibitors; and proteins, polypeptides and peptides.

A number of proteins and peptides have a potential for being suitable for inhalation therapy and some of them are in various stages of development. Some examples are insulin, alpha-1-proteinase inhibitor, interleukin 1, parathyroid hormone, genotropin, colony stimulating factors, erythropoietin, interferons, calcitonin, factor VIII, alpha-1-antitrypsin, follicle stimulating hormones, LHRH agonist and IGF-I, Ketobemidone, Fentanyl, Buprenorfin, Hydromorfon, Ondansetron, Granisetron, Tropisetron, Scopolamine, Naratriptan, Zolmitriptan, Almotriptan, Dihydroergotamine, Somatropin, Calcitonin, Erythropoietin, Follicle stimulating hormone (FSH), Insulin, Interferons (alfa and beta), Parathyroid hormone, alfa-1-antitrypsin, LHRH agonists, vasopressin, vasopressin analogues, desmopressin, glucagon, corticotropin (ACTH), gonadotrophin (luteinizing hormone, or LHRH), calcitonin, C-peptide of insulin, parathyroid hormone (PTH), human growth hormone (hGH), growth hormone (HG), growth hormone releasing hormone (GHRH), oxytocin, corticotropin releasing hormone (CRH), somatostatin analogs, gonadotropin agonist analogs (GnRHa), human gatrial natriuretic peptide (hANP) recombinant human thyroxine releasing hormone (TRHrh), follicle stimulating hormone (FSH), and prolactin.

Other possible polypeptides include growth factors, interleukins, polypeptide vaccines, enzymes, endorphins, glycoproteins, lipoproteins, and polypeptides involved in the blood coagulation cascade, that exert their pharmacological effect systemically.

Powder engineering has advanced in recent years. As stated above, in the field of dry powders for inhalation it is customary to formulate pharmaceutically active substances with carrier particles of inert material such as lactose. The carrier particles are designed such that they have a larger mean aerodynamic diameter than the active substance particles making them easier to handle and store. The smaller active agent particles are bound to the surface of carrier particles during storage, but are torn from the carrier particles upon actuation of the device. This process is often referred to as de-agglomeration. In order to assist in the de-agglomeration is has been proposed to employ so-called force-controlling agents or anti-adherent additives in admixture with active and carrier particles. Force controlling agents can be surface active materials. By judiciously selecting the type and amount of force-controlling agents employed it is possible to manipulate the amount of force required to remove the active particles from the carrier particles. One such force-controlling agent is magnesium stearate. Dry powder formulations employing magnesium stearate and the like are described in U.S. Pat. No. 6,645,466. This patent is hereby incorporated by reference, and devices containing powders disclosed in this patent represent particularly preferred embodiments of the present invention. Other force-controlling agents or anti-adherent additives are described in U.S. Pat. No. 6,521,260, which is herein incorporated by reference.

The following is a description by way of example only with reference to the accompanying drawings of a particular embodiment of the present invention. The detailed structure of this embodiment, its construction and operation are described in detail in U.S. Pat. No. 6,182,655. The following discussion and drawings therefore describe details of the embodiment only relating to the means for bifurcating an air-flow passing through the device and any other parts of the device that needs to discussed in order to understand such means.

In the drawings:

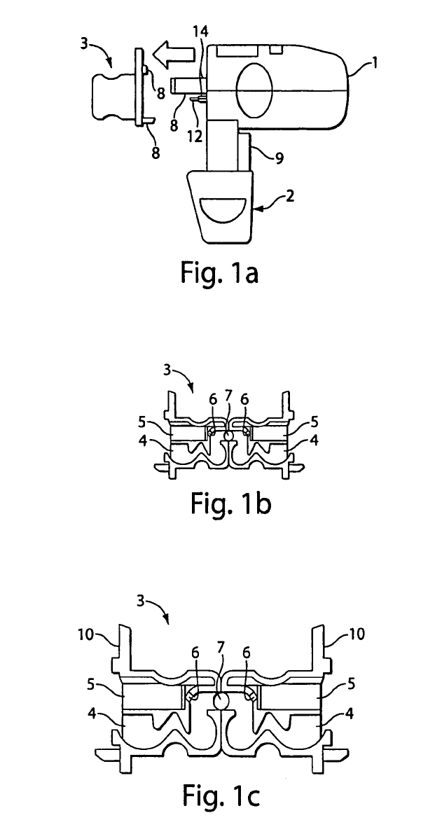

FIG. 1a is a side view of a device and a cap (disengaged).

FIG. 1b is a depiction of a mouth-piece for use in the embodiment of FIG. 1a, showing the mouth-piece split along a longitudinal plane of symmetry and the two parts hinged apart through 180 degrees.

FIG. 1c is an enlarged view of FIG. 1b.

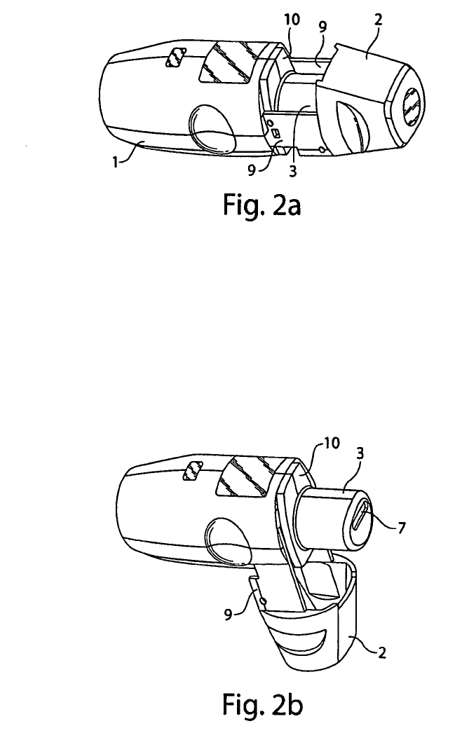

FIG. 2a is a perspective view of the device wherein the cap is in a state of opening

FIG. 2b is a perspective view of the device wherein the cap is fully opened

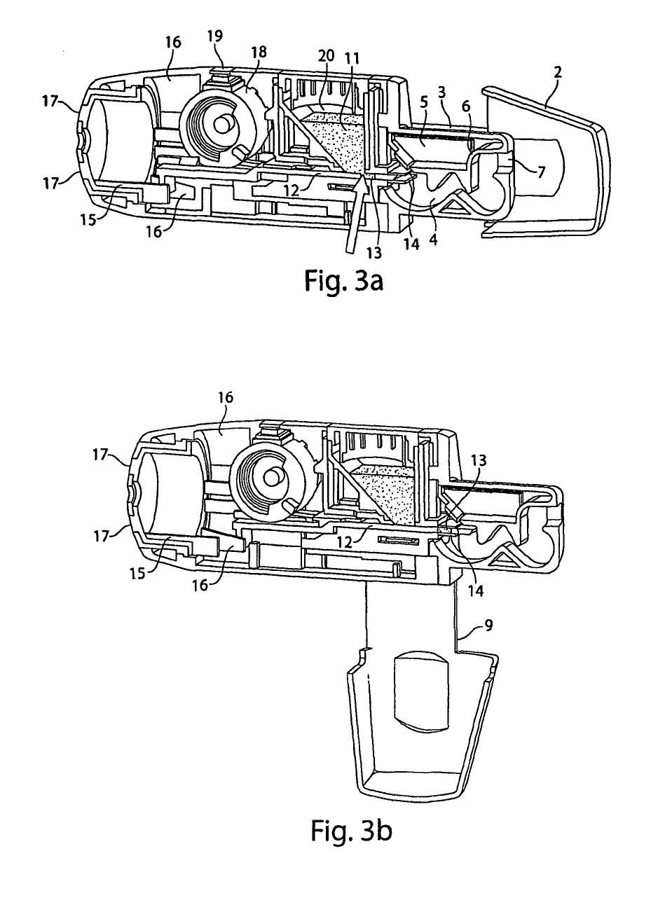

FIG. 3a shows a cross-section of the device wherein the cap is in a state of opening

FIG. 3b shows a cross-section of the device wherein the cap is fully opened

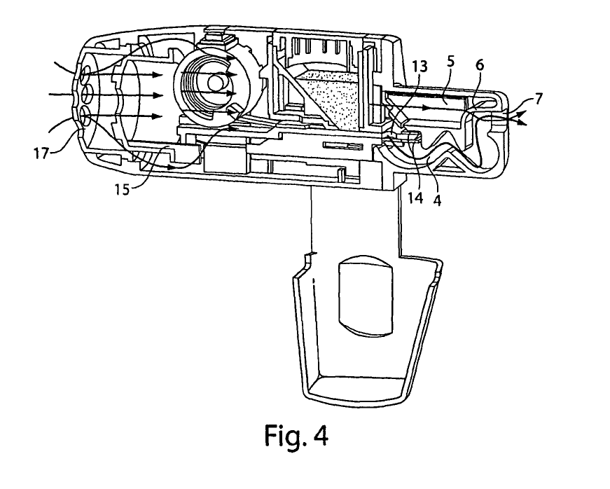

FIG. 4 shows a cross-section of the opened device and schematically shows how an air-flow is generated through the device upon breath-actuation by a user

With reference to FIGS. 1a and 1b, a device according to the present invention comprises an elongate body (1), and a cap (2) that is connected to the body by arms (9) such that it is slidably moveable outwards away from the body and rotationally downwards of the body guided by the arms (9). Mouth-piece (3) is adapted to fit onto the body by means of projections (8) provided on the mouth-piece and the body. When body and mouth-piece are disengaged it is possible to see a dosing arm (12) associated with a dispensing chamber (not shown) in dispensing position, and a shutter (14) that is slidable over said dispensing chamber and which covers the dispensing chamber in said dispensing position.

FIG. 1 b shows the mouth-piece formed of two hinged halves that are closable to form the mouth-piece. The mouth-piece contains a first channel (4) and a second channel (5) that are in fluid communication with an exit port (7). The first channel is adapted to receive a first portion of an air-flow entrained in which is a powder dispensed from the dispensing chamber. The second channel (5) is adapted to receive a second portion of the air-flow, which does not contain any entrained powder. In addition to receiving a portion of the air-flow, channel (5) is adapted to receive and support projection (8) contained on the body (1). At one end of channel (5) there is an orifice (6) which permits the portion of air-flow passing therethrough to re-combine with the air-flow through the first channel (4) before exiting the exit port (7). The mouth-piece (3) comprises a back-plate (10) that abuts the body (1) when mouth-piece and body are engaged.

FIG. 2a is a perspective view of the device in which the cap (2) is in a first stage of opening whereby the cap is pulled away from the body whilst being guided by arms (9) that are slidably mounted on guide rails (not shown) internal of the body. FIG. 2b shows the device in perspective when it is fully opened to present the mouth-piece to a patient. The arms (9) are connected by hinges (not shown) internal of the body, which permit the cap to be rotated into its fully open position. The patient is able to place its mouth over mouth-piece (3), and inhale with a minimum inspiratory effort in order to inhale entrained powder through the mouth-piece (7).

FIG. 3a shows a cross-section view of the device as shown in FIG. 2a. In FIGS. 3a and 3b, arms (9) are connected to a dosing slide (12) such that movement of the cap (2) is translated through the arms (9) to the dosing slide (12) which in turn moves a dispensing chamber (13) containing a discrete dose of powder (11) contained in a powder reservoir (20) from a position directly under the reservoir in the direction of the mouth-piece. When the cap is in a fully closed position the dispensing chamber is located directly under the reservoir (20), but it moves in the direction of the cap movement as the latter is removed. The Bold arrow shows the dispensing chamber (13) in a position intermediate between its receiving position under the reservoir and its dispensing position. FIG. 3b shows the cap (2) moved rotationally about the body (1). With the cap in its fully opened position, the dosing slide (12) has moved forward to its maximum extent and has positioned the dispensing chamber (13) proximate to the first channel (4) in the mouth-piece. The dispensing chamber is now in its dispensing position. In its dispensing position, the dispensing chamber is located inside the shutter (14), which covers both the bottom and top of the chamber there to retain the powder (11) in the chamber (13). At the same time, a valve shield (15) is in its closed position and covers openings (17) in the body (1).

In FIG. 4 a user (not shown) places its mouth over the mouth-piece (3) and inhales with a minimum inspiration effort. This effort creates an under pressure in the body of the device actuating the valve shield (15) and moving it into its forward position. The valve shield arms (16) are likewise driven forward into contact with abutment portions (not shown) connected to the shutter (14) which communicates the movement of the valve shield to the shutter and urges the latter forward and away from the dispensing chamber. At the same time, the air inlets (17) are opened as the valve-shield moves into its forward position allowing an air-flow (see arrows) to move through the device and across the dispensing chamber thereby to entrain the powder (11). The entrained powder is directed through the channel (4) wherein it is de-agglomerated by the air turbulence created in the tortuous passage. At the same time, a portion of the air flow passes through the channel (5). As the dispensing chamber is located proximate to the entrance of the channel (4) no, or substantially no, entrained powder passes through the channel (5). Air-passing through the channel (5) and through the orifice (6) re-combines with the air-flow through channel (4) before passing through the exit port (7).

Once the inhalation process is complete, the cap can be closed and a returning mechanism (not shown) moves the dosing slide such that the dispensing chamber once again sits under the reservoir in its receiving position and the valve shield returns to its start position. The counter mechanism (18) records a correctly administered dose and this can be seen by a user through a recording window (19).

There now follows an example that illustrates the advantages of the present invention.

### EXAMPLE 1

In this example, the fine particle fraction (mean aerodynamic diameter of 4.4 microns or less) as a percentage of the dose emitted from a dry powder inhaler according to the present invention as compared to a device bearing a mouth-piece having a single channel containing de-agglomerating means as described in U.S. Pat. No. 6,182,655 was measured.

Measurements were made using a Dosage Unit Sampling Apparatus (DUSA). The apparatus therefore are well known in the art, and are described in the United States Pharmacopoeia 24 Chapter <601>.

The apparatus was operated at variable air-flow rates, in each case to produce a pressure drop of 4 kPa over the particular dry powder inhaler tested.

A comparator device according to U.S. Pat. No. 6,182,655 having a single channel through the mouth-piece gave a delivered dose of 8.8 micrograms and a fine particle fraction (less than or equal to 3.3 microns) of 36.8% (RSD of FPF of delivered dose=0.5%; n=3).

Four devices identical to the comparator but for their having the 2 channel mouth-piece of the present invention were tested.

The tested devices differed only in the diameter of the orifice (6) in the second channel.

Device 1 (having an exit port diameter of 1.5 mm) gave a delivered dose of 8.8 micrograms with a fine particle fraction of 41.2% (RSD of FPF of delivered dose=1.5%; n=3).

Device 2 (2.0 mm) gave a delivered dose of 8.4 micrograms with a fine particle fraction of 39.0% (RSD of FPF of delivered dose=6.6%; n=3).

Device 3 (2.5 mm) gave a delivered dose of 8.2 with a fine particle fraction of 36.9% (RSD of FPF of delivered dose=3.0%; n=3).

Device 4 (3.0 mm) gave a delivered dose of 7.9 with a fine particle fraction of 34.8% (RSD of FPF of delivered dose=3.3%; n=3).

In all cases, the reported fine particle fraction relates to those particles having mean aerodynamic diameter of less than or equal to 3.3 microns.

The resistance through the comparator device and the 4 devices according to the present invention were tested on the same apparatus as mentioned above.

The comparator device was tested at 54.3 L/min to obtain a pressure drop of 4 kPa. The resistance was measured at 0.118.

Device 1 (orifice 1.5 mm) was tested at 55.5 L/min to obtain pressure drop of 4 KPa. The resistance was measured at 0.116.

Device 2 (orifice 2.0 mm) was tested at 62.5 L/min to obtain pressure drop of 4 KPa. The resistance was measured at 0.103.

Device 3 (orifice 2.5 mm) was tested at 65.2 L/min to obtain pressure drop of 4 KPa. The resistance was measured at 0.098.

Device 4 (orifice 3.0 mm) was tested at 70.9 L/min to obtain pressure drop of 4 KPa. The resistance was measured at 0.090.

The results demonstrate that the devices of the present invention give statistically similar results in terms of the fine particle fraction of the delivered dose as the comparator device, although the resistance through the devices of the invention is lowered significantly.

1. A DPI device comprising a dispensing chamber for receiving a discrete dose of medicament-containing powder and means for delivering said dose from the dispensing chamber to a patient in an air-flow that passes from the chamber to the patient via a mouth-piece along a first air passage that comprises de-agglomerating means, the device additionally comprises a second air passage in fluid communication with the dispensing chamber and the mouth-piece and which by-passes the de-agglomerating means and which is located such that it receives a portion of said air-flow that is free, or substantially free, of powder.

2. A DPI device according to claim 1 comprising:
a dispensing chamber for receiving a discrete dose of medicament-containing powder;
means for delivering said dose on an air-flow from the dispensing chamber to a patient via a mouth-piece; said means for delivering a dose comprising an air passage in fluid communication between dispensing chamber and exit port, which is adapted to channel said air-flow through the device;
means for de-agglomerating a powder disposed along the air passage; and
means for bifurcating the air-flow into first and second portions, the first portion containing entrained powder that passes through the de-agglomerating means before being dispensed from an exit port in the mouth-piece, the second portion containing no entrained powder and by-passing the de-agglomerating means before being dispensed from the mouth-piece.

3. A DPI device according to claim 1 or claim 2 wherein the device is breath-actuated.

4. A breath-actuated inhaler device according to claim 3 for dispensing a dose of powder to the respiratory tract of a patient, which device comprises:
a hollow housing containing an air inlet, and valve means in releasable sealing arrangement with said air inlet, the valve means being actuated to allow an air-flow to pass through an air passage in the housing in response to a pre-determined minimum inspiratory effort of a patient;
a dispensing chamber internal of the housing for receiving a discrete dose of medicament-containing powder; and
a mouth-piece attached, or adapted to be attached, to said housing, said mouth-piece comprising: a hollow body having at a first end an exit port through which a powder can pass into the respiratory tract of a patient, and at a second end an opening in communication with the air-passage of the device when the mouth-piece is connected to the housing; and first and second channels bringing said exit port and opening into fluid communication, wherein said first channel describes a tortuous air passage for de-agglomerating a powder, and wherein upon actuation said air-flow is bifurcated such that a first portion of the air-flow containing the entrained powder passes through the first channel and through an exit port into the respiratory tract of the patient, and a second portion of said air-flow not containing entrained dry powder passes through the second channel of the mouth-piece and through an exit port in the mouth-piece and into the respiratory tract of the patient.

5. A breath-actuated inhaler device according to claim 3 for dispensing a powder to the respiratory tract of a patient comprising:
a hollow housing having openings at opposed ends, having a mouth-piece attached to the housing at a first end and valve means positioned inside the housing and covering a second end, the mouth-piece having first and second channels extending therethrough, said first channel describing a tortuous path for de-agglomerating powder entrained on an air-flow;
a protective cap covering said mouth-piece, said cap being moveable between an closed position covering the mouth-piece and an open position exposing the mouth-piece for use by a patient;
a powder reservoir located inside the housing and including a funnel outlet;
a moveable dosing mechanism located inside the housing containing a dispensing chamber, said dispensing chamber being moveable between a receiving position directly under said funnel outlet, and a dispensing position proximate to the first channel of the mouth-piece when the cap is moved from closed to open position;
a slidable cover covering said dispensing chamber when in its dispensing position; said valve means being moveable between a rest position sealing one opposed end opening, and a forward position in response to a defined minimum inspiratory effort during inhalation by a patient, said valve shield moving said slidable cover from over the dispensing chamber when moving into its forward position to permit an air-flow external of the device to enter the housing thereby to entrain the powder such that a first portion of said air-flow containing entrained powder passes through the first channel of the mouth-piece before passing through an exit port in the mouth-piece and into the respiratory tract of a patient, and a second portion of the air-flow not containing entrained dry-powder passes through the second channel and through an exit port in the mouth-piece to be inhaled by a patient;
returning means for returning said dispensing chamber to the receiving position after inhalation by a patient; and
recording unit positioned inside the housing, said recording unit recording the number of inhalation operations performed.

6. A mouth-piece adapted to releasably attach to an inhaler device comprising:
a hollow body comprising: at one end an exit port through which a dry powder can pass into the mouth of a patient, and at the other end an opening through which an air-flow emitted from the inhaler device may pass; and means for releasably attaching the mouth-piece to an inhaler device; the hollow body having running therethrough a first channel describing a tortuous air passage for de-agglomerating a powder passing through the channel entrained on a first portion of said air-flow; and
a second channel running therethrough, through which a second portion of said air-flow passes not containing entrained powder.

7. A device according to claim 3 wherein the powder contains an active substance useful in treating conditions of the lung selected from the group consisting of asthma or chronic obstructive pulmonary disease (COPD); or useful to treat systemic disease states via delivery through the lung.

8. A device according to claim 7 wherein the active substance is selected from β2-adrenoreceptor agonists; steroids; anticholinergic bronchodilators; mucolytics; antihistamines; cyclooxygenase inhibitors; leukotriene synthesis inhibitors; leukotriene antagonists; phospholipase-A2 (PLA2) inhibitors; platelet aggregating factor (PAF) antagonists; prophylactics of asthma; antiarrhythmic medicaments; tranquilisers; cardiac glycosides; hormones; antihypertensive medicaments; antidiabetics; anti-allergic medicaments; expectorants; antiparasitic medicaments; anticancer-medicaments; sedatives; analgesic medicaments; antibiotics; antirheumatic medicaments; immunotherapies; antifungals; antihypotension medicaments; vaccines; antiviral medicaments; vitamins; anti-oxidants; free-radical scavengers; COX II inhibitors; NSAIDS; PDE4 inhibitors and PDE5 inhibitors; proteins; polypeptides; and peptides.

9. A device according to claim 6 or claim 7 wherein the active substance is selected from the group consisting of salbutamol, terbutaline, rimiterol, fenoterol, reproterol, adrenaline, pirbuterol, isoprenaline, orciprenaline, bitolterol, salmeterol, formoterol, clenbuterol, procaterol, broxaterol, picumeterol, TA-2005, mabuterol, budesonide, ciclesonide, mometasone, fluticasone, beclomethasone, flunisolide, loteprednol, triamcinolone, amiloride and rofleponide, ipratropium bromide, sodium cromoglycate, nedocromil sodium, insulin, celecoxib α-1-proteinase inhibitor, interleukin 1, parathyroid hormone, genotropin, colony stimulating factors, erythropoietin, interferons, calcitonin, factor VIII, α-1-antitrypsin, follicle stimulating hormones, LHRH agonist and IGF-I, Ketobemidone, Fentanyl, Buprenorfin, Hydromorfon, Ondansetron, Granisetron, Tropisetron, Scopolamine, Naratriptan, Zolmitriptan, Almotriptan, Dihydroergotamine, Somatropin, Calcitonin, Erythropoietin, follicle stimulating hormone (FSH), Insulin, Interferons (α and β), Parathyroid hormone, α-1-antitrypsin, LHRH agonists, vasopressin, vasopressin analogues, desmopressin, glucagon, corticotropin (ACTH), gonadotrophin (luteinizing hormone, or LHRH), calcitonin, C-peptide of insulin, parathyroid hormone (PTH), human growth hormone (hGH), growth hormone (HG), growth hormone releasing hormone (GHRH), oxytocin, corticotropin releasing hormone (CRH), somatostatin analogs, gonadotropin agonist analogs (GnRHa), human gatrial natriuretic peptide (hANP) recombinant human thyroxine releasing hormone (TRHrh), follicle stimulating hormone (FSH), prolactin, growth factors, interleukins, polypeptide vaccines, enzymes, endorphins, glycoproteins, lipoproteins, polypeptides involved in the blood coagulation cascade, and their pharmacologically acceptable esters and salts.

10. A medicament for inhalation into the lung comprising a unit dose or multiple unit doses of a powder containing an active substance useful in treating conditions of the lung selected from the group consisting of asthma or chronic obstructive pulmonary disease (COPD); or useful to treat systemic disease states via delivery through the lung contained in a device of claim 3.

11. A method of de-agglomerating dry-powder from a DPI device prior to its inhalation by a patient, the method comprising:
generating an air-flow for entraining a powder;
bifurcating said air-flow into a first portion containing entrained powder, and a second portion not containing dry powder;
channeling said first portion through a mouth-piece and into the respiratory tract of a patient via means for de-agglomerating dry powder; and
channeling said second portion through the mouth-piece and into the respiratory tract of a patient by-passing said de-agglomerating means.
